(19) United States
(12) Patent Application Publication
(10) Pub. No.: US 2008/0319411 A1
(43) Pub. Date: Dec. 25, 2008

(54) ADHESIVE UNDERGARMENT

(75) Inventors: WILLIAM MORTENSEN, Plano, TX (US); CELINDA SOLANO, Arlington, TX (US); KRISTIN AARON, Bedford, TX (US)

Correspondence Address: HOWISON & ARNOTT, L.L.P, P.O. BOX 741715, DALLAS, TX, 75374-1715, US

(73) Assignee: ADVANCED MATERIALS, INC., Dallas, TX (US)

(21) Appl. No.: 11/767,137

(22) Filed: Jun. 22, 2007

(51) Int. Cl. A61F 13/15

(52) U.S. Cl. 604/387; 604/358

(57) ABSTRACT

An adhesive undergarment to be worn when panties are not worn. Embodiments of an adhesive undergarment may have an adhesive-backed panel for placement inside a garment. An exemplary adhesive undergarment provides a level of protection to the private area of a woman from the interior seams of pants or other similar clothing when being worn without panties. This clothing accessory is in the nature of a removable, disposable, adhesive-backed panel for placing inside the crotch area of a garment that replaces a need for a panty. An adhesive undergarment may have a strengthened area or a stiffened area to minimize the drooping, flopping or wilting of the adhesive undergarment while being installed in the crotch area of a pair of pants.

### TECHNICAL FIELD

Embodiments of the present invention relate to an adhesive-backed panel for placement inside a garment that replaces a panty. More particularly, embodiments of the invention relate to a clothing accessory in the nature of removable, disposable, adhesive-backed panel for placement inside the crotch area of a garment that replaces a need for a panty.

### BACKGROUND

A visible panty line (VPL) occurs when the outline of someone's underpants or panty becomes visible through his or her clothes. There are various causes for VPLs. VPLs may be caused by the outer clothing being partially transparent due to the nature of the fabric being a thin silk, thin cotton, or thin satin fabric. The fabric may have been exposed to water making it transparent or clingy such that a VPL is apparent. The lighting conditions about the person wearing pants or shorts may reveal VPLs. The VPLs may be made apparent when a darker colored underwear or panty is being worn underneath lighter-colored clothing. Furthermore, tight, clingy outer-clothing, such as leggings or stretch pants may reveal VPL. Circumstances wherein clothing is made of soft fabric, such as in jogging pants or business pants, reveal VPLs when the wearer is sitting or bending over thereby making indentations or seams of the panty or underwear show through the pants, shorts or skirt material.

Visible panty lines are almost entirely a female issue due to the types of clothing and gender roles of males and females. Generally, there is a taboo in many cultures against showing ones underwear. Such a taboo has been extended to panty lines, such that VPL are generally considered undesirable and embarrassing.

In the recent past, panties and lingerie products were specifically designed such that VPL are non-existent or not visible. Thongs, T-backs, G-strings, boy shorts, and a variety of other women's panty styles have entered the market place, all for eliminating visible panty lines. Furthermore, in the recent past, it has become fashionable for some women to wear pants without any undergarments underneath. The act of not wearing any undergarments under outer-clothing is known as “going pantiless,” or “going commando”.

In order to provide some protection for women who go pantiless, temporary, disposable patches or liners may be used for placement in the crotch area of pants, shorts, exercise clothing, or other garments that a user may wear without panties. Presently, crotch liners for such situations face various technical challenges. Such crotch-liners must be comfortable to user when being worn for extended periods of time. Such crotch-liners should be made for easy installation in the crotch area of a garment without creating any uncomfortable or abrasive folds or ridges that may rub against the wearer's skin or private parts in the crotch area. Also, if the crotch-liner is an adhesive-type liner, the liner, when the adhesive is peeled from the bottom-side of the liner, should not fold upon itself such that the adhesive sticks to another portion of the adhesive. If the adhesive side of a crotch liner sticks to itself it may ruin the adhesive strength of the crotch-liner or render the crotch liner unusable. Thus, there are various technological challenges presented in the design and manufacture of crotch-liners for use in garments for when the user is pantiless.

### SUMMARY

In some embodiments of the invention, a removable adhesive pant liner is provided. The removable adhesive pant liner is intended for placement on the crotch and inseam area on the inside of a pair of pants, shorts, exercise clothing or other garments when garment wearer does not want to wear an undergarment or have visible panty lines. In some embodiments, the removable adhesive pant liner comprises a fabric layer that is generally longer in a longitudinal direction than in a lateral direction. A first adhesive layer is on the bottom side of the fabric layer. A paper layer is on the bottom side of the first adhesive layer. A second adhesive layer is on the bottom side of the paper layer. And, a removable release layer is on the bottom side of the second adhesive layer. There may be an alignment aide or indicator that is visible on the top side of the fabric layer. Such an alignment indicator may be located in more than one position in order to aid the alignment of the removable adhesive pant liner with one or both of the crotch seam and the inseam inside a garment. Some embodiments comprise a stiffening region that provides temporary stiffness to the removable adhesive pant liner when a release layer (sometimes referred to as a release liner) is removed from the bottom side of the second adhesive layer. The stiffening region aids the user in placement of the removable adhesive pant liner inside the crotch of a pair of pants by minimizing an ability of the removable adhesive pant liner from folding over or creasing upon itself such that the second adhesive layer sticks to itself. After the removable adhesive pant liner is installed and worn, the stiffening region becomes ineffective so that the embodiment moves with the garment fabric.

In other embodiments of a removable adhesive pant liner, the fabric layer is made of a knit fabric having elasticity in the longitudinal, the lateral, and a combination of longitudinal and lateral directions so as to allow the embodiment to move with the fabric of the garment it is adhered to.

In yet other embodiments of a removable adhesive pant liner, the fabric layer is made of translucent fibers or threads such that markings or graphics on the paper layer can be seen through the fabric layer.

Furthermore and in various embodiments, the fabric layer is made of adsorbent fiber components, rather than absorbent fiber components, such that the fibers or threads making up the fabric layer do not swell or become enlarged when moistened or wet.

In yet other embodiments the paper layer may comprise a graphic design on the top side or bottom side thereof. Furthermore, the paper layer may be comprised of tissue paper or a derivation thereof. In yet other embodiments, the graphic design may comprise a stiffening material, such as a polymer or starch or sizing material. The stiffening material may temporarily stiffen the embodiment as explained above.

In yet other embodiments, the removable adhesive pant liner may include embossed portions wherein channels are formed in the removable adhesive pant liner thereby establishing a stiffening effect in the removable adhesive pant liner prior to being installed in the crotch area of a garment. The embossed portions may establish graphics, designs or be merely utilitarian in nature.

In yet other embodiments, a cover layer is adhered to or removably placed on the top side of the fabric layer. The cover layer may act as a stiffening agent to maintain a stiffness of the removable adhesive pant liner after the release layer is removed from the bottom side of the second adhesive layer. The removable cover also may aid the user in placing embodiments of the removable adhesive pant liner in the crotch area of a pair of pants such that the removable adhesive pant liner does not fold upon itself, wrinkle or crease when being applied to the garment. Furthermore, in embodiments wherein the removable cover layer completely covers the top side of the fabric layer, the removable cover layer acts as a fabric layer contamination barrier.

These and other features found in embodiments of the removable adhesive pant liner will become more apparent from the following description and figures.

### BRIEF DESCRIPTION OF THE DRAWINGS

For a more complete understanding of the various embodiments of the invention and the features thereof, reference is now made to the following Detailed Description taken in conjunction with the accompanying Drawings in which:

FIG. 1 illustrates a top view of an exemplary removable adhesive pant liner;

FIG. 2 illustrates a side view of an exemplary removable adhesive pant liner;

FIG. 3 illustrates a perspective exploded view of an embodiment of the removable adhesive pant liner;

FIGS. 4A-4C illustrate exemplary fabric layers of a removable adhesive pant liner;

FIGS. 5A-5D illustrate exemplary first adhesive layers of an exemplary removable adhesive pant liner;

FIGS. 6A-6C illustrate exemplary paper layers of an exemplary removable adhesive pant liner;

FIGS. 7A-7C illustrate exemplary second adhesive layers that may be found in embodiments of a removable adhesive pant liner;

FIGS. 8A-8C illustrate exemplary release layers of embodiments of removable adhesive pant liners;

FIG. 9 illustrates an exemplary removable cover layer for some embodiments of a removable adhesive pant liner;

FIG. 10 illustrates a bottom view of an embodiment wherein a portion of the release layer is being peeled away; and

FIG. 11 illustrates an embodiment of the invention being installed in the crotch area of a pair of pants.

### DETAILED DESCRIPTION OF THE EXEMPLARY EMBODIMENTS

Referring now to the drawings, wherein like reference numbers are used herein to designate like elements throughout the various views, embodiments of the present invention are illustrated and described, while other possible embodiments of the present invention are described. The figures are not necessarily drawn to scale, and in some instances the drawings have been exaggerated and/or simplified in places for illustrative purposes only. One of ordinary skill in the art will appreciate the many possible applications and variations of the present invention based on the following examples of possible embodiments of the present invention.

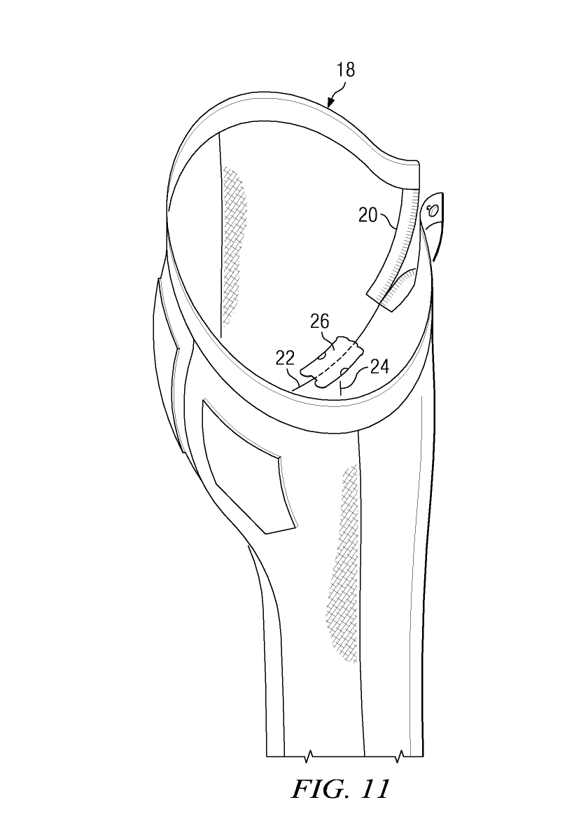

Referring first to FIG. 10, an embodiment a removable adhesive pant liner 10 is shown. FIG. 10 is a bottom view showing a release layer 12 that peels off in the direction of arrow 14. An adhesive 16 is exposed as the peel-off layer 12 is removed. The removable adhesive pant liner 10 may then be aligned and placed within a pant. Referring to FIG. 11, a pant 18 is shown. This particular pant 18 has a front 20 with a fly or button closure. A crotch seam 22 is found extending from the front 20 of the pants 18 to the rear of the pants 18. An inseam 24 extends substantially perpendicular to the crotch seam 22. Various pant garment types can be used with exemplary embodiments of the invention.

An embodiment of the invention 10 may be placed on the crotch seam 22 of pants 18. The upper surface 26 of the exemplary removable adhesive pant liner 10 is made of a knit fabric. The exemplary removable adhesive pant liner 10 may be placed, after a first part of the release layer 12 has been removed, on the fabric on one side of the crotch seam as shown in FIG. 11. The second part of the release layer 28 can be then peeled off from the bottom of the adhesive pant liner 10, such that the adhesive pant liner 10 may then be adhered to the fabric on the other side of the crotch seam 22.

A feature provided by some embodiments of the removable adhesive pant seam is protection of the user's private area from abrasion or other roughness produced by the inseam and crotch seam when the user is wearing the pants 18 without panties. Furthermore, the removable adhesive pant liner is thin enough that panty lines or edge lines of the exemplary embodiment cannot be seen or are at least minimally visible through the fabric of the pants 18. Another feature of the exemplary removable adhesive pant liner 10 is that the knit fabric 26 of the upper surface is made of non-absorbent fibers, such as nylon, Lycra® or other reasonable equivalents. The non-absorbent fibers are instead adsorbent and, therefore, do not swell like absorbent cotton fibers when moistened or wet. Furthermore, the surface tension, weave, and various layers of an exemplary removable adhesive pant liner 10 minimize any moisture seepage through the embodiment 10 to the pant's crotch area. The adhesive layer 16 is strong enough to hold an embodiment 10 in position in the crotch area of a pair of pants 18 without peeling or having the edges of the embodiment roll up, thereby causing an abrasion or irritation to the user's skin in the area. Furthermore, the adhesive layer 16 is weak enough that the embodiment can be removed from the crotch area of the pair of pants 18 with a pulling or peeling action by the user. Embodiments may be disposed of after the pants 18 have been worn and prior to washing the garment.

Thus, some features of exemplary embodiments are that there are no panty lines created by the embodiment. If any adhesive pant liner lines are created by an embodiment, the lines will be minimal and be located in the crotch area of the pants 18 where they cannot be easily seen while the user is wearing the pants 18. Furthermore, another feature of various embodiments is that it is a pant accessory that is easy to peel and place into position within the crotch area of a pair of pants. This is most useful for a user who wishes to wear tight or snug-fitting knit pants, shorts, jeans, or exercise wear. Embodiments of the invention may be worn once and then disposed of. They may be made of a combination of nylon and Lycra® such that they are smooth and comfortable to sensitive crotch areas of the user.

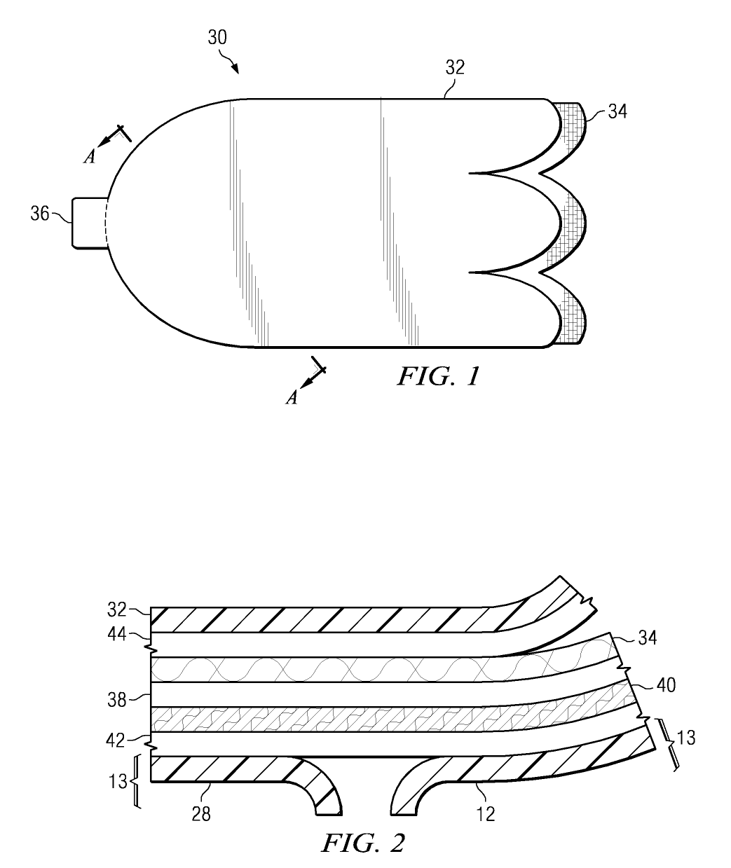

Referring now to FIG. 1, another embodiment of a removable adhesive pant liner 30 is shown. FIG. 1 is a top view of a pant liner showing a removable stiffening layer 32 positioned on the top of a fabric layer 34. The upper removable stiffening layer 32 may be made of paper, a composite of paper, a polymer or plastic sheet that provides some minimal amount of stiffness to the pant liner 30 when the release layer portions 12, 28 are removed from the bottom of the pant liner 30. It has been found that without a stiffening layer 32, embodiments of the invention may fold over upon themselves when the release layer portions 12 and 28 have been removed. If the bottom adhesive portion of the pant liner touches itself and sticks to itself, it weakens the bottom adhesive layer of the pant liner 30, thereby potentially weakening the hold of the entire pant liner on the crotch area of a pair of pants 18 when an exemplary pant liner 10, 30 is installed. Thus, the removable stiffening layer 32 provides some level of stiffness to help stop the fabric 34 and/or the plurality of layers from wilting or folding over upon itself while the embodiment 30 is being installed and placed in the crotch area of a pair of pants. A tab 36 that extends beyond the periphery of the fabric layer 34 may be included on the removable stiffening layer 32 to aide the user to more easily remove the removable stiffening layer 32 after the adhesive pant liner 30 is installed in the crotch area of a pair of pants.

Referring now to FIG. 2, the various layers that make an exemplary removable adhesive pant liner 30 are shown in a side or cut-away view A from FIG. 1. Fabric layer 34 may be made of a woven or knit material. In an exemplary embodiment, the fabric layer 34 is made of knitted material that is a nylon-Lycra® blend. The thickness of the fabric layer 34, from top side to bottom side, is from about 0.2 mm to about 1 mm. The fabric layer 34 is stretchable in any planar direction. The flexibility and stretchability of the fabric layer 34 is an important feature such that the embodiment will move easily with the pants or other garment to which it is adhered. If the fabric layer 34 was not flexible and stretchable in substantially any direction, then the exemplary embodiment may show puckers or unusual lines through thin satin or silk fabrics in the crotch area of the user during normal user movements.

In some embodiments, the fabric layer 34 has a nylon-Lycra® blend of between 75% and 98% nylon and between about 2% and about 25% Lycra®. After experimentation by the inventors with various blends of nylon and Lycra®, it was determined that this range provides the most comfort and smoothness to the user when an embodiment is worn on the interior of a garment's crotch. Other fibers may be blended with the nylon and Lycra®.

A first adhesive 38 is applied to or on the bottom side of the fabric layer 34. A paper layer 40 is adhered to the bottom side of the first adhesive layer 38. The paper layer 40 comprises a very thin paper or a type of tissue paper. Paper may be described in several ways, and the International Organization for Standardization (ISO) paper industry standard is considered the most consistent way to compare and define types of paper. The ISO compares and defines paper by its weight. The ISO measures weight in grams per square meter (GSM). The ISO defines tissue paper as being in the range of 10-35 GSM.

A second adhesive layer 42 is applied to the bottom side of the paper layer 40. The second adhesive layer 42 has a weaker adhesive strength than the first adhesive layer 38. A reason for the first adhesive layer 38 being stronger than the second adhesive layer 42 is so when the exemplary removable adhesive pant liner 30 is removed from a garment, the stronger first adhesive layer 38 will stop the fabric layer 34 from pulling away from or separating from the paper layer 40. The weaker second adhesive layer 42 stays adhered to the paper layer 40 and will not tend to leave residual adhesive on the garment when an exemplary removable adhesive pant liner is being removed therefrom. The weaker second adhesive layer 42 adheres better to the paper layer 40 than to most garment fabrics.

On the bottom side of the second adhesive layer 42 is a release layer 13. The first release layer portion 12 covers a first portion of the second adhesive layer 42 and the second release layer portion 28 covers the remaining portion of the second adhesive layer 42. As such, the release layer portions 12 and 48 completely cover the second adhesive layer 42. The release layer 13 may be in one piece or in a plurality of portions. The release layer 13 may be any known release layer used on stickers or adhesive strips and is to be peeled away therefrom. The release layer may be made of a paper composite, plastic polymer material, or any other material known for such use in the art. The release layer 13 provides a stiffness to the pant liner 30 such that it is easy to hold and manipulate the liner into position. Generally, one portion of the release layer, for example release layer portion 12, is removed from an exemplary removable pant liner prior to the liner being adhered to the crotch portion of a user's pair of pants. Once the first portion (e.g., 26) of the adhesive pant liner 30 is attached to the user's pants 18, the second release layer, for example release layer 28, may be removed. The remaining portion of the second adhesive layer is uncovered and made available for adherence to the crotch portion of a user's pants.

In some embodiments, a removable stiffening layer 32 may be on top of the fabric layer 34. A third adhesive layer 44 may be disposed on all of or a portion of the bottom side of the removable stiffening layer 32 to gently adhere the removable stiffening layer 32 to the fabric layer 34. The removable stiffening layer 32 and third adhesive layer 44 may completely cover the fabric layer 34. By completely covering the fabric layer 34, the removable stiffening layer and adhesive layer provide a barrier against contamination of the fabric layer 34. In some embodiments, the fabric layer 34 is hypoallergenic or sterilized.

In yet other embodiments, the removable stiffening layer 32 and third adhesive layer 44 may partially cover the top side of the fabric layer 34 such that some stiffening of the removable adhesive pant liner is created and maintained when the release layer 13 is removed. By providing the stiffening to the adhesive pant liner 30, it has been found that the thin exemplary adhesive pant liner is easier to install over the crotch seam 22 of a pair of pants 18 without creating bumps, ridges, or other potentially irritable lines in an exemplary removable adhesive pant liner 30. Furthermore, once installed, the stiffening layer 32 and third adhesive layer 44 are peeled off of the fabric layer 34 providing a clean, smooth fabric surface that will be near or against the user's body while the garment 18 is being worn without panties thereunder.

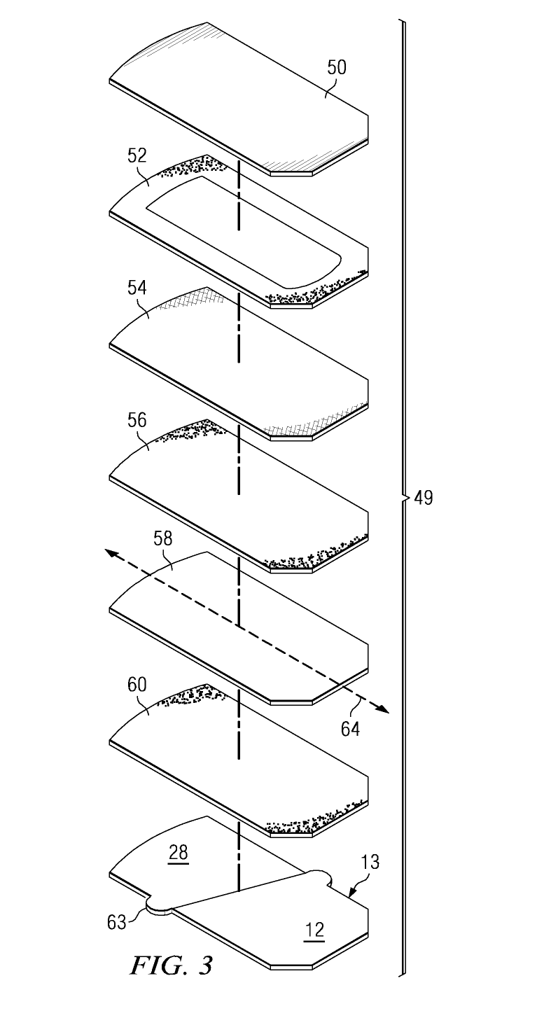

Referring now to FIG. 3, a perspective exploded view of an exemplary removable adhesive pant liner 49 is depicted. On the very top of an exemplary removable adhesive pant liner 49 is a removable stiffening layer 50. As discussed above, the removable stiffening layer 50 may either partially or completely cover the top of the fabric layer 54. A third adhesive layer 42 is adhered to all or a portion of the bottom side of the removable stiffening layer 50. The third adhesive layer 52, in combination with the removable stiffening layer 50, operate together to adhere the stiffening layer to the top of the fabric layer 54. The combination of the removable stiffening layer 50 and the third adhesive layer 52 may or may not be a necessary part of an exemplary embodiment due to the fact that an exemplary removable adhesive pant liner may have different stiffening means incorporated therein as will be explained below. The removable stiffening layer 50 and third adhesive layer 42 may be peeled from the fabric layer 54 with ease such that none or a negligible amount of the adhesive layer remains adhered to the fabric layer 54. Substantially all of the adhesive layer 42 remains intact on the bottom surface of the removable stiffening layer 50. The stiffening layer 42 may be made of paper, a paper composite, a plastic/polymer sheet, a silicon sheet or other thin material that adds a degree of stiffness to an embodiment's overall structure.

As discussed above, the combination of the removable stiffening layer 50 and third adhesive layer 52 may provide a barrier against dirt and other foreign matter that are not welcome on the fabric layer 54 prior to use. In some embodiments, the fabric layer's hypoallergenic or sterilized features are somewhat protected by incorporating the removable stiffening layer 50 and third adhesive layer 52 thereon. The removable stiffening layer 50 provides a stiffness to the overall exemplary removable adhesive pant liner 49 when the release layers 12 and 28 are removed and the removable adhesive pant liner 49 is being installed in the crotch of a pair of pants 18 by a user. In some embodiments, the removable stiffening layer 50 does not require an adhesive to stay removably secured to the fabric layer. For example, a silicon, latex, polyethylene, PVC, or other polymer or non-polymer based removable stiffening layer 50 may removably adhere to the top of the fabric layer 54 without requiring the addition of a separate adhesive therebetween.

Fabric layer 54 may be made of a wide variety of materials including, but not limited to, nylon, Lycra®, cotton, Spandex®, polyester, silk, satin, or substantially any combination of thread or fabric blends that may be used in women's panty or stretchable exercise clothing. Through experimentation it has been determined that a very acceptable and comfortable fabric blend for use in embodiments is a nylon-Lycra® fabric blend fabric layer 54. The positive features of such a fabric blend is that it is smooth and soft to the touch and does not chaff or irritate the sensitive body parts or skin areas that are being protected from the potentially irritable crotch and inseam seams of a garment. Furthermore, the fabric layer 54 can be woven or knitted to be extremely thin in the range of about 0.1 to about 1 mm in thickness. A fabric layer that is between 0.1 and 1 mm thickness is advantageous because it will tend not to create a line or outline when adhered to the thinnest of women's silk, satin, or Spandex® garments. The fabric layer 54 may have a nylon percentage of from 75%-98% nylon and a Lycra® percentage from between 2% and 25% Lycra®. In some embodiments of the invention, the fabric layer 54 is a 92% nylon/8% Lycra® blend. In yet other embodiments of the invention, has been found that an 84% nylon/16% Lycra® blend is also very comfortable. The fabric layer 54 may be woven, but it has been found that a knit fabric is more desirable. A circular knit fabric providing elasticity or resilient stretch in multiple directions or substantially all directions of potential fabric movement provides the best movement with the garment fabric. Although, some embodiments may provide a fabric layer that is absorbent, it has been found that an adsorbent fabric that holds very little moisture and does not swell when moist provides a more comfortable and better adhered removable adhesive pant liner because less chaffing, unraveling and better elasticity and movement with the garment resulted than with absorbent materials. In various embodiments, the fabric layer 54 may be knit or woven to have graphics or designs incorporated therein. The fabric layer 54 may be a solid color, such as black, beige or light blue, but may also be so thin or made with translucent thread, such that the overall fabric layer 54 has a translucent quality. A translucent, very thin embodiment of a removable adhesive pant liner will be difficult to detect from the exterior of a thin, clingy garment wherein the user is going pantiless. The graphic or design may be for advertising or other public relations related reasons.

A first adhesive layer 56 is applied to or on the bottom side of the fabric layer 54. As will be discussed below, the first adhesive layer 56 may or may not completely cover the bottom side of the fabric layer 54. The first adhesive layer 56 may cover a peripheral area, linear sections, random spots or be in an organized or random pattern on the bottom side of the fabric layer 54. Under the first adhesive layer 56, there is a paper layer 58. The combination of the fabric layer 54, first adhesive layer 56, and the paper layer 58 provide a feature wherein a liquid is not necessarily repelled from the top surface of the fabric layer 54, but also does not tend to soak through the fabric layer 54 on to a garment that the exemplary removable adhesive pant liner is adhered to. The paper layer 58 may be a tissue paper or derivation thereof such that its weight per square meter is between 10 and 35 GSM. The tissue paper may be hypoallergenic, or sterile. On the bottom side of the paper 58, a second adhesive layer 60 is applied. In some embodiments the paper layer 58 has a weight that is greater than 35 GSM, but less than about 65 GSM. In such embodiments, the thickness of the paper layer 58 also act as a stiffening means to help prevent an embodiment from being too floppy or folding over upon itself when the release layer 13 is removed.

The second adhesive layer 60 is generally applied substantially covering the bottom side of the paper 58; however, in some embodiments, the second adhesive layer 60 may be applied in stripes that extend longitudinally 64 the length of the exemplary removable adhesive pant liner. Furthermore, the second adhesive layer 60 may be applied in stripes that are lateral to the longitudinal direction 64 of the removable adhesive pant liner 49. A longitudinal center-line 64 is shown on the paper layer 58 in FIG. 3. The longitudinal center-line line 64 is generally a line that would be aligned by the user with the garment's crotch seam when applying an embodiment to the inside of the pair of pants 18. In some embodiments a marking or alignment means visually indicates the longitudinal center line 64 to aid the user when placing an embodiment in a garment.

The second adhesive layer 60 has a lower adhesive strength than the first adhesive layer 56. The second adhesive layer 60 has a stronger adhesive strength than the third adhesive layer 52. The first adhesive layer 56, second adhesive layer 60 and third adhesive layer 52 all are selected to minimize a potential for irritation or an allergic reaction when placed against the crotch area of the user for extended periods of time.

Release layers 12 and 28 are adhered to the bottom side of the second adhesive layer 60. Release layers 12 and 28 are removable by the user prior to installing an exemplary removable adhesive pant liner 49 to the crotch and inseam portions of a pair of pants. The release layers 12 and 28, in some embodiments, may only be a single peel-off layer 13, rather than two separate pieces. Furthermore, a tab 63 may extend beyond the periphery of the paper layer 58 and fabric layer 54 to aid the user in peeling off the release layers 28.

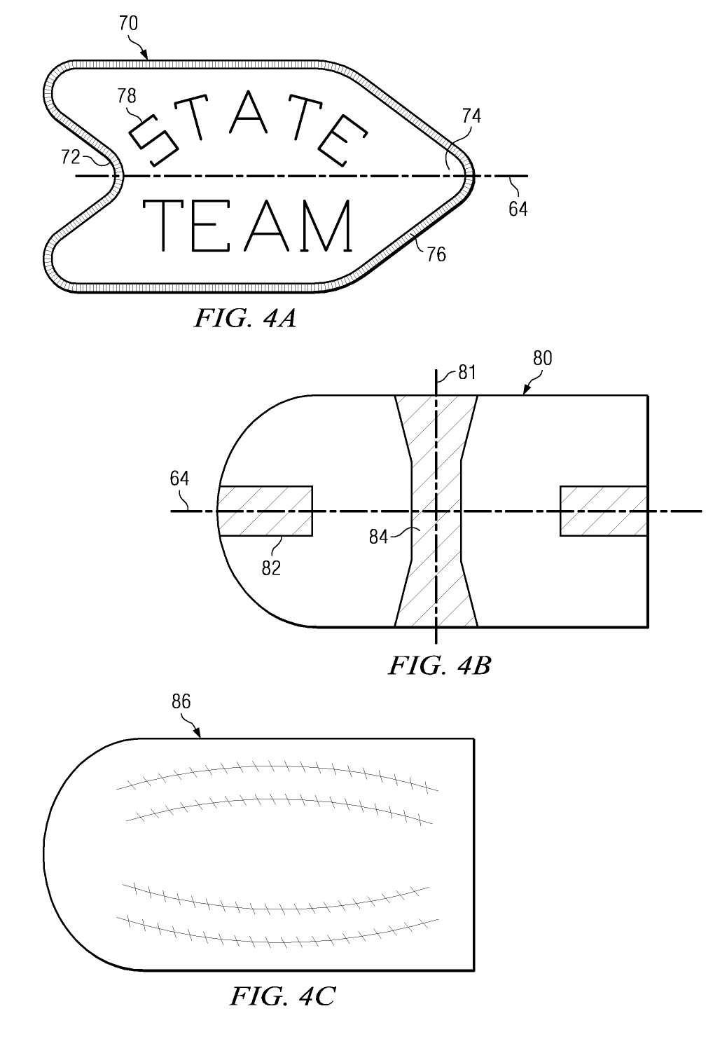

Referring now to FIGS. 4A, 4B, and 4C, the fabric layer of various embodiments is described in more detail. FIG. 4A depicts a fabric layer 70. A first crotch seam alignment aide 72 is depicted as a notched portion and a second crotch seam alignment aide 74 in the form of an alignment arrow or pedal is shown at the longitudinal 64 opposite side of the exemplary fabric layer 70. The crotch seam alignment aide 72 and 74 aid a user in aligning an exemplary removable adhesive pant liner with the crotch seam inside a garment. Furthermore, the periphery of the fabric layer 70 comprises edge finishing 76. The edge finishing 76 may be an embossed periphery, a heat-treated or partial melt of the fabric layer periphery, an edge seam, a polymer outline, a starch, ink, glue, or other type of edge finishing that provides a means for minimizing raveling of the threads woven in the fabric layer along the periphery thereof. Furthermore, in some embodiments, the edge finishing 76 provides a stiffening means that aids in maintaining the shape of an exemplary removable adhesive pant liner when the bottom-most release layer (i.e., release layer 13) is removed.

Furthermore, a design or logo 78 may be embossed into the fabric layer 70. In other embodiments, the graphic, design, or logo 78 may be established via a partial-melt, ultrasonic melt, applique, embroidery of, weave-in of, printing on, or lasering of the fabric layer 70. The feature of embossing or partially melting or applying or incorporating other graphics into the fabric layer 70 provides a means for incorporating additional stiffness into the fabric layer to aid the user in placing the removable adhesive pant liner in the crotch of a pair of pants when either of the release layers [62A] or [62B] are removed therefrom. The incorporated stiffness may be insignificant after a removable adhesive pant liner is installed in a garment. Embossing provides indentions that create grooves or channels in the fabric layer that temporarily strengthen the fabric, but becomes insignificant when installed in a pair of pants.

FIG. 4B depicts another exemplary fabric layer 80 that has alignment indicia or aides embossed partially heat-melted, or incorporated thereon. A first alignment aide 82 extends down the longitudinal center-line of the fabric layer 80. Another alignment aide could be a lateral line 81 that is embossed, heat-melted or incorporated perpendicular to the longitudinal center-line 64 to help a user align a removable adhesive pant liner with the inseam inside a pair of pants. Another alignment aide 84 may be a letter, part of a graphic emblem or graphic that would aid the alignment of the removable adhesive pant liner with either the inseam or the crotch seam in a pair of pants.

FIG. 4C shows yet another embodiment of the fabric layer 86 having a graphic design embossed, partially heat-melted or otherwise incorporated onto the fabric layer 86 to provide a stiffening means for stiffening the overall removable adhesive pant liner when the release layer has been removed by the user prior to installing the pant liner into a garment.

It should be understood that when embossing is used to establish a stiffening means, the embossing in some embodiments may only be applied to one specific layer, for example, the fabric layer 86 or the paper layer 40. In other embodiments, the embossing may be applied to more than one layer, such as a combination of the fabric layer and paper layer. Still yet in other embodiments, the embossing may be performed such that most or all of the layers are embossed.

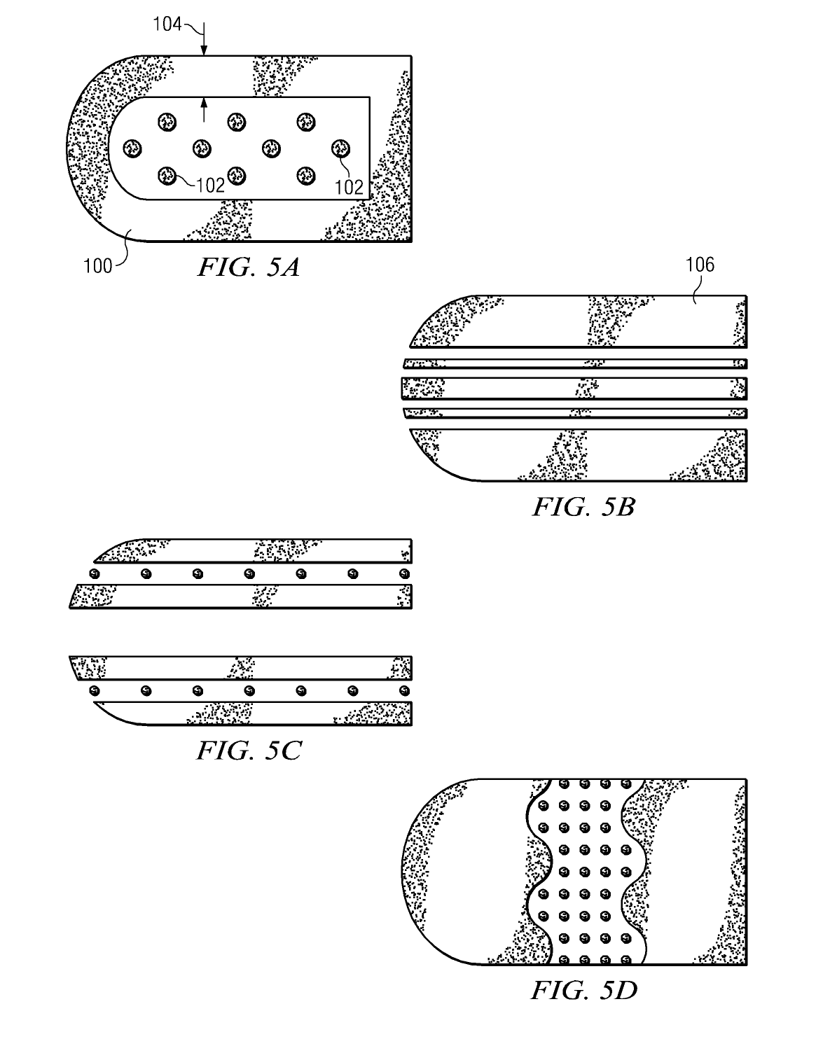

Referring now to FIGS. 5A, 5B, 5C, and 5D, embodiments of the first adhesive layer is depicted. FIG. 5A depicts one embodiment of the first adhesive layer wherein a predetermined width 104 of adhesive is provided about the periphery of the fabric layer. The predetermined width 104 of adhesive about the periphery 100 may or may not be complimented with small portions 102 of additional adhesive positioned in an orderly or random fashion within the periphery of the peripheral adhesive layer 100. The feature of not providing a complete layer of adhesive in a central location of the bottom side of fabric layer allows the removable adhesive pant liner to breathe and perhaps aid or enable some evaporation of moisture through the embodiment. In another embodiment of the first adhesive layer shown in FIG. 5B, the adhesive layer 106 is provided in longitudinal strips that are of different widths or the same widths. The longitudinal strips of adhesive provide a strong adhesive strength for holding fabric layer and the paper layer together, but may provide some non-adhesive areas on the fabric area to aid in aeration and/or evaporation of moisture through the removable adhesive pant liner. In various embodiments of the invention, it is important to have the first adhesive substantially along the peripheral edges of the first adhesive layer so that the embodiment does not roll or peel away from the garment while being installed and being worn by the user. FIGS. 5C and 5D depict additional exemplary configurations of a first adhesive layer. The adhesive layer may be applied in a longitudinal direction or lateral direction. The adhesive layer may be applied in parallel strips, in squiggly lines or in random or organized spotted locations. One feature of an embodiment is to have intermittently applied adhesive or no adhesive applied in the central area of embodiments of the invention. By minimizing the application of adhesive in the central portion, embodiments of the invention may be allowed to breathe a bit and/or provide evaporation of moisture through the pant liner. The first adhesive may cover from 0% to 100% of a central area, of about 1 in.×1 in., of the fabric layer.

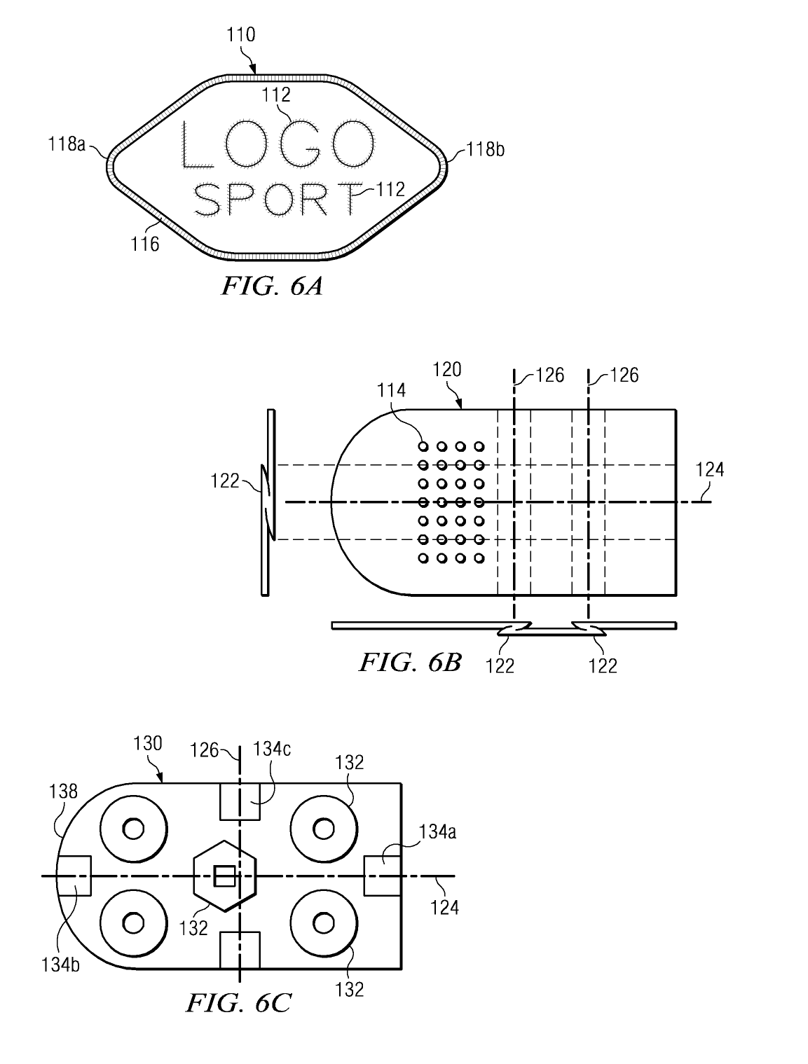

Referring now to FIGS. 6A, 6B and 6C, discussion of the paper layer will be provided. The paper layer, as described above, is preferably of tissue paper weight and is of substantially the same shape and format of the fabric layer. In FIG. 6A, an embodiment of a paper layer 110 is provided wherein a logo or graphic 112 is printed on either the topside or bottom side of the paper layer 110.

FIGS. 6A, 6B and 6C, depict embodiments of the paper layer as it may be incorporated into embodiments of an exemplary removable adhesive pant liner. Referring first 6A, the paper layer 110 is a tissue weight paper material. Furthermore, the paper layer 110 may be a tissue weight woven material or reasonable facsimile or derivation of a tissue weight paper material. The tissue layer 110 is usually substantially identical in shape to its fabric layer counter part. The paper or tissue layer 110 can be made of a paper product but in other various embodiments the paper layer 110 may be made of a polymer, plastic or other chemically manufactured material. The paper layer may also be a mesh or screen-like fabric, perforated paper product, or non-woven air-permeable material. The paper layer may also be made of a silk, knit or other fabric woven material. The tissue layer 110 provides a separation between the first adhesive layer and the second adhesive layer such that the first adhesive layer, which has a stronger adhesive strength than the second adhesive layer, will remain adhered to both the fabric layer and the paper layer 110 when an exemplary removable adhesive pant liner is removed from the fabric of the garment. The second, weaker, adhesive layer adheres to the paper layer 110 with a greater adhesive strength than it adheres to the fabric so that when the removable adhesive pant liner is removed from a garment the second adhesive layer will leave a minimum of adhesive residue with the garment. In some embodiments, the paper layer comprises a breathable or porous layer. In other embodiments the paper layer is porous, perforated or screen-like and allows the first adhesive to adhere not only to the paper layer but also to contact and adhere therethrough to the second adhesive in the second adhesive layer. Such adhesion will strengthen the second layers adhesion to the paper layer when an embodiment is removed from a garment. Such perforations or porosity 114 is showing in a small portion of FIG. 6b for illustrative purposes.

In some embodiments, a logo 112 may be printed on the paper layer 110. The logo 112 may be printed on either the top side, the bottom side or both sides of the paper layer 110. If the fabric layer is somewhat translucent or transparent, the logo 112 will be visible through the fabric layer. If the fabric layer is opaque, the logo 112 may have a thickness visible as waves or bumps on the top side of the fabric layer. Such a logo may be used for advertising, team or school logos, the product name or brand, a desired graphic design or words. The graphic may be personalized for the particular user or for a holiday or season.

In other embodiments, the graphic or logo 112 may be printed with a polymer-based, silicon-based, or starch-based ink such that the logo adds a degree of stiffness to the tissue paper layer 110 and results in being a stiffening means for the overall removable adhesive pant liner. Furthermore, ink or embossing can be placed about the periphery 116 of the tissue paper layer 110. The peripheral printing or embossment 116 may comprise a stiffening means in the form of a thick-colored ink, non-colored ink or embossed creases, folds, or edge designs about the edges of the paper layer. In some embodiments an additional layer of paper or other stiffening material may be added to provide a stiffening beam, stiffened, or other stiffening or strengthening means. Since the paper layer 110 is substantially the same shape as the fabric layer 34, 70, a crotch seam alignment aide 118a and 118b may be included at either end of the paper layer 110. The alignment aide 118a,b, may aid the user in aligning an exemplary removable adhesive pant liner with a crotch seam in the pants or garment that the exemplary pant liner is being installed in. In FIG. 6A, the alignment aides 118a,b, are a blunt or pointed tab bump, or marker that may be used to visually and physically align the longitudinal center-line of the embodiment with a crotch seam.

FIG. 6B shows a 3-view of another embodiment of a paper layer 120 wherein the paper layer 120 is reinforced via a z-fold 122. The 3-view depicts how a z-fold 122 is essentially a folding of the paper layer in a z-format, both along the longitudinal center-line 124, and in some embodiments, along a lateral line 126 that is perpendicular to the longitudinal center-line 124. The z-fold may operate as a stiffening means of stiffening beam that adds a strengthened beam or stiffness region(s) to the overall removable adhesive pant liner when being installed by a user.

In yet another embodiment of the invention shown in 6C the paper layer 130 is depicted with a stiffening means 132 thereon or therewith. Here the stiffening means 132 may be concentric circles or offset circles that are printed with a thick polymer ink or starch-like substance to stiffen the paper layer 130. The stiffening means may further be embossed channels, having u-shaped, v-shaped or other shaped channels, formed by embossing the surface of the tissue layer 30 or the entire embodiment. The embossing of the tissue layer may take place prior to the tissue layer being combined with the other layers of an exemplary removable adhesive pant liner or be embossed after the fabric layer, first adhesive layer, paper layer, second adhesive layer and release layer have been assembled. The stiffening means of various embodiments of the invention may function like a beam, support structure or stiffening means that holds the fabric and adhesive layers against the forces of gravity and/or static electricity that may make the removable adhesive pant liner flop, be limp or attempt to stick to itself or fold over when all or part of the release layer is removed from the embodiment. This is important for some embodiments of the invention because a user is not interested in wasting an exemplary crotch liner due to a malfunction (e.g. folding and sticking to itself or a bumpy or creased installed embodiment) of the crotch liner if it becomes wilted or limp while the user is installing the removable adhesive pant liner in a pair of pants.

Still referring to FIG. 6C, there is an alignment aide 134a adjacent to the front edge 136 of the paper layer 130. Another alignment aide 134b may be located on the back edge 138 of the paper layer 130. Yet another alignment aide 134c may be positioned proximate to one or both lateral edges of the paper layer 130. The alignment aides 134a-c may be seen through a somewhat transparent or translucent fabric layer of an exemplary removable adhesive pant liner. The alignment aides 134a-c may be used by a user to aid in the alignment of an exemplary removable adhesive pant liner along the crotch seam and/or inseam of a pair of pants or garment.

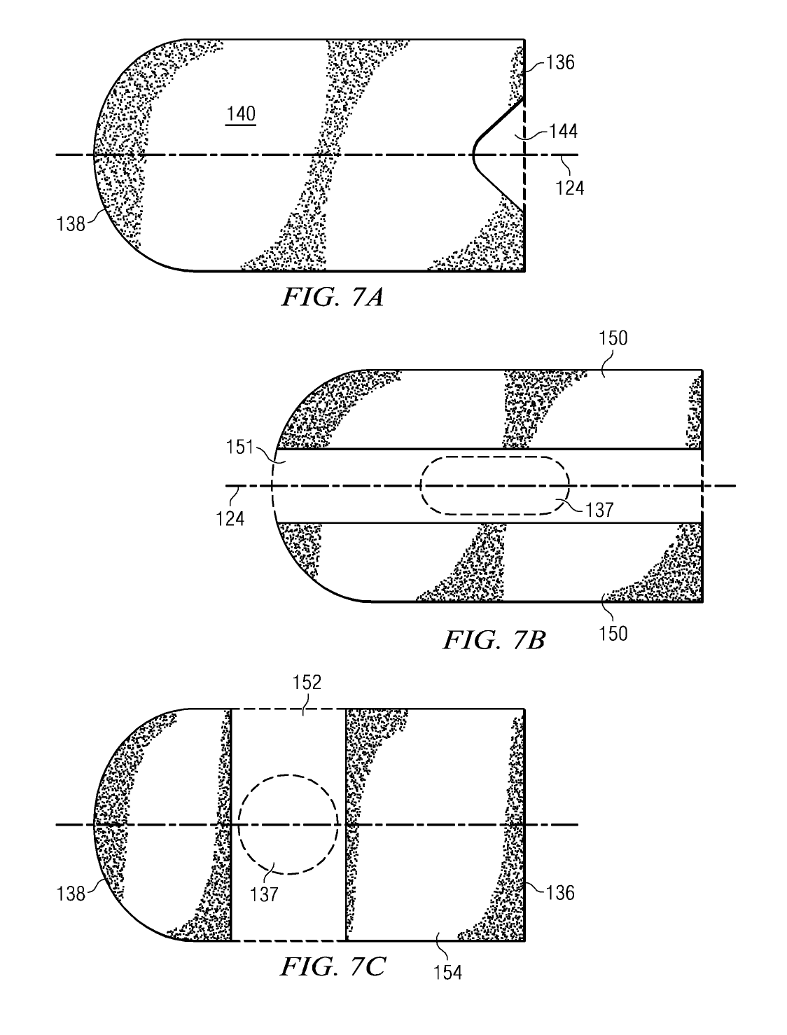

Referring now to FIGS. 7A, B and C, embodiments of the second adhesive layer will be discussed. In FIG. 7A, a second adhesive layer 140 is shown that would cover substantially all of the bottom surface or side of an exemplary paper layer. The second adhesive 140 has a lower adhesive strength than the first adhesive discussed above. In some embodiments a fingertip size area, being 0.5 to 3 square cm in area, may not have second adhesive in the second adhesive layer. Such a fingertip sized non-adhesive area 144 may be adjacent to the outer edge or perimeter of the fabric layer or second adhesive layer 140 such that a user's fingertip can be placed, at least partially, underneath the fabric layer when an exemplary removable adhesive pant liner is attached to a user's garment. The user's fingertip may be placed in the non-adhesive area 144 such that the removable adhesive pant liner may be easily gripped and removed from a garment or pants by the user. In some embodiments the non-adhesive area 144 may be located at one or both ends of an exemplary embodiment along the longitudinal center-line 124. It has been found through experimentation that positioning the non-adhesive area 144 in the second adhesive layer along the longitudinal center-line 124 at either the front 136 or the back 138 of the removable adhesive pant liner greatly aids the user in removing an exemplary removable pant liner. It is understood that such a non-adhesive 144 area may be placed substantially anywhere or in a plurality of locations about the periphery of the second adhesive layer 140.

Referring now to FIG. 7B, another embodiment of the second adhesive layer is shown. This adhesive layer 150 is divided into parallel strips of adhesive extending in a longitudinal direction and parallel with the longitudinal center-line 124. In some embodiments there are a plurality of second adhesive strips extending longitudinally. In other embodiments, two longitudinal strips of second adhesive extend parallel to the longitudinal center-line 124. The two second adhesive strips of adhesive are positioned to be spaced apart such that they do not cover the center portion 137 of the paper layer. The non-adhesive area 151 of the second adhesive layer may extend the full longitudinal length down the center-line of the second adhesive layer in some embodiments.

Referring to FIG. 7C yet another embodiment of the second adhesive layer provides adhesive in the front that is adjacent to the front 136 of the second adhesive layer and the back 138 of the second adhesive layer, but leaves a non-adhesive area 152 at least in a central portion 137 of the second adhesive layer 154, without adhesive. One reason for not applying second adhesive in the central portion 137 of the second adhesive layer is to aid the breathability of embodiments of a removable adhesive pant liner. Furthermore, by not having second adhesive in the central area of embodiments of the invention, evaporation of moisture may occur. The breathability or evaporation may take place through the garment that embodiments of the invention are installed on. In yet other embodiments the second adhesive layer is substantially similar in configuration to the first adhesive layer.

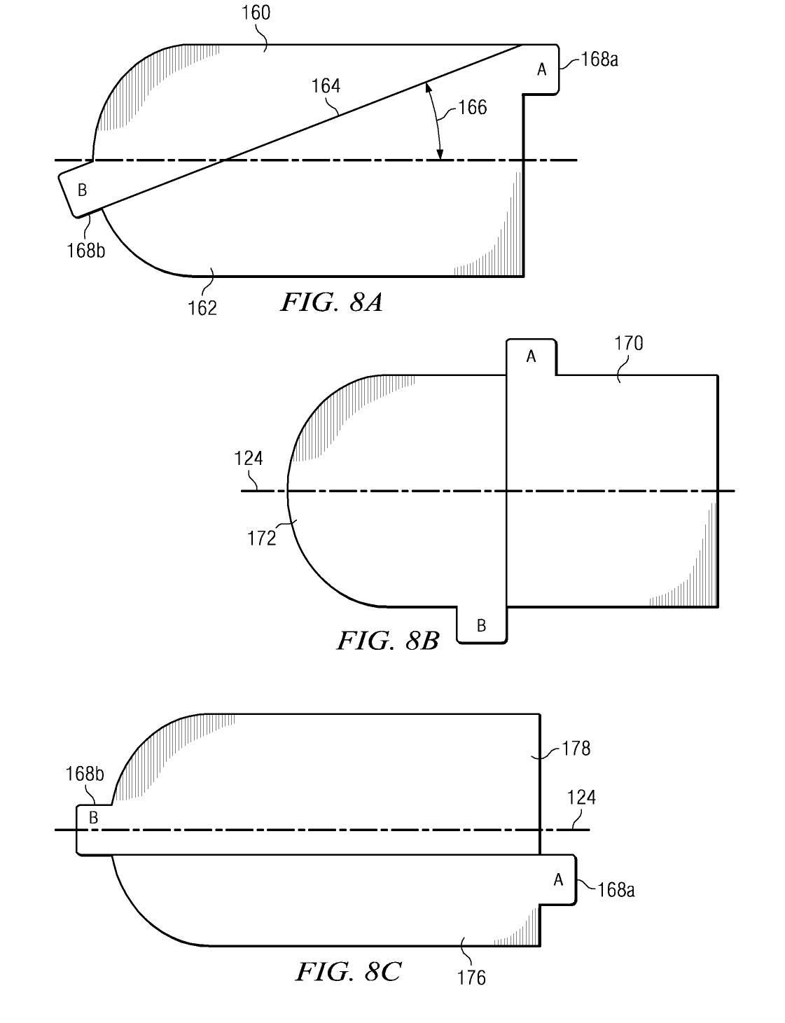

Referring now to FIGS. 8A, B and C, the release layer of various embodiments are depicted. FIG. 8A shows a release layer having two parts. A first part 160 and a second part 162. Both release layer parts 160 and 162 are adjacent to each other and meet at a line 164 that is diagonal to or at an angle 166 with the longitudinal center-line 124 of an exemplary embodiment. A release layer tab 168a and 168b are provided, one for each release layer. Each release layer tab 168a,b, extends beyond the peripheral edge of the fabric or paper layers of embodiments of the invention. The release layer tabs 168a,b, provide a grip means or a physical location for a user to hold, peel and remove the release layer from the second adhesive layer discussed above. In FIG. 8B, a two part release layer is shown having a first part 170 and a second part 172 that are adjacent to each and separated by a lateral dividing line that is perpendicular to the longitudinal center-line 124. FIG. 8C depicts yet another embodiment of the release layers showing a two part release layer 176 and 178 wherein the two release layer parts cover the second adhesive layer and are divided by a release layer dividing line 180 that is parallel and offset from the longitudinal center-line 124. In other embodiments not specifically shown, a single release layer may completely cover the bottom of an exemplary embodiment or merely cover the portions on the bottom of the exemplary embodiment wherein a second adhesive exists. That is, if the second adhesive layer covers a portion or portions of the bottom side of the paper layer, then the release layer may cover only the second adhesive layer portions. Regardless of whether the release layer covers just the second adhesive layer portions or more than the second adhesive layer portions on the bottom side of the paper layer there still may be grip tabs 168a and 168b for aiding the user in grasping the release layer to remove it from the second adhesive layer.

The release layer may be coated, at least on the top side, with a substance, known as the sticker art, such that the release layer does not readily stick with a significant amount of adhesive strength to the second adhesive layer.

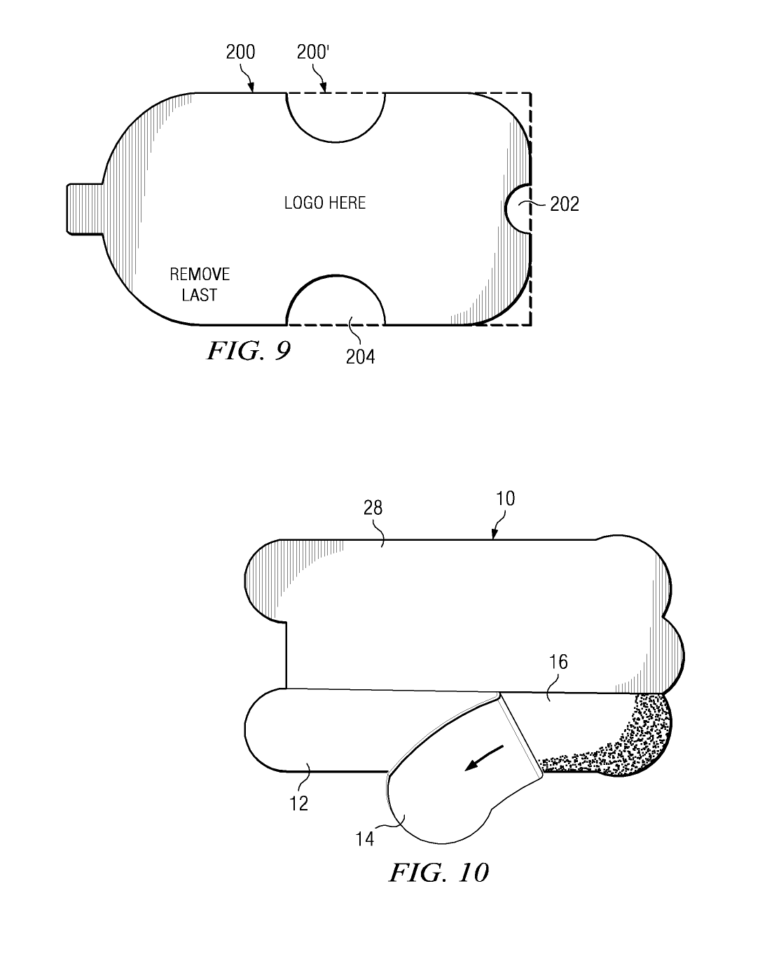

Refer now to FIG. 9; a removable top stiffening layer, found in some embodiments is shown. The removable top stiffening layer 200 may completely cover the fabric layer as depicted with the solid and dotted peripheral lines in FIG. 9 or only cover a portion of the fabric layer as shown as the partial top stiffening layer 200. The stiffening layer may have printing or graphics thereon for advertising the embodiment, other products, schools, colleges or sport teams. The top stiffening layer may also comprise alignment aides 202, 204 in the form of tabs, notches, or graphics to aid in aligning embodiments with the crotch and/or inseams inside a pair of pants. The top stiffening layer 200 or 200′ is generally removed after the release layer has been removed from an embodiment and after the embodiment is adhered, second adhesive side down, to the crotch portion of a garment. The stiffening layer may be part of the stiffening means or be the stiffening means by itself. The stiffening layer 200, 200′ stops the fabric from flopping, wilting or adhering to itself after the release layer has been removed. The stiffening layer may further aide a user in aligning and placing an embodiment of the invention on their garment such that the fabric layer is less likely to bunch, crease, fold over or be placed and installed incorrectly within the garment.

Referring again to FIGS. 10-11, the release layer 12 may be removed or peeled from the embodiment 14 such that an embodiment 10 can be placed in the crotch section of a pair of pants 18 on the crotch seam 22 and inseam 24. Embodiments of the invention may be used in place of panties such that the user may wear a garment without panties underneath yet obtain some protection from abrasive crotch seams and potential moisture stains. The use of an embodiment enables a user to wear a tight fitting or loose fitting pant garment in such a manner such that panty lines are not seen through the garment due to there being no panties underneath the garment. Once installed in the garment the user may comfortably wear the garment with confidence that the garment will lie smooth against the wearer's body and visible panty lines will not be seen. Furthermore, any rough inseams or crotch seams are covered by embodiments of the invention in the area of the user's private parts to thereby aid in minimizing bruising or rashes caused by rough or unfriendly inseam or crotch seams while the garment is being worn.

Embodiments of the invention are generally disposable after being removed from a worn garment.

The overall thickness of an exemplary embodiment without the release layer and removable stiffening layer is less than a millimeter. In general, the average overall thickness of the fabric adhesive and paper layers is between 0.1 and 0.9 mm thick, preferably less than 0.5 mm thick.

It will be appreciated by those skilled in the art having the benefit of this disclosure that embodiments of this invention provide an adhesive undergarment that may be worn in the crotch area of a pair of pants, shorts or exercise clothing and that provides a level of protection for the wearer's private area from abrasion and general pain that may be caused by a rough inseam or crotch seam in a garment. Embodiments of the invention are to be worn when panties or other undergarments are not being worn under a pair of pants or shorts or exercise clothing and provide a means for eliminating panty lines. It should be understood that the drawings and detailed description herein are to be regarded in an illustrative rather than a restrictive manner, and are not intended to limit the invention to the particular forms and examples disclosed. On the contrary, the invention includes many further modifications, changes, rearrangements, substitutions, alternatives, design choices, and embodiments apparent to those of ordinary skill in the art, without departing from the direction and scope of this invention, as defined by the following claims. Thus, it is intended that the following claims be interpreted to embrace all such further modifications, changes, rearrangements, substitutions, alternatives, design choices, and embodiments.

What is claimed is:

1. A removable adhesive pant liner comprising:
a fabric layer having a longitudinal direction and a lateral direction;
a first adhesive on a bottom side of said fabric layer;
a paper layer on a bottom side of said first adhesive layer;
a second adhesive layer on a bottom side of said paper layer;
a removable release layer on a bottom side of said second adhesive layer;
an alignment indicia visible on a top side of said fabric layer;
a longitudinal and lateral direction stiffening region providing stiffness to said removable adhesive pant liner when said release layer is removed from said 10 bottom side of said second adhesive layer.

2. The removable adhesive pant liner of claim 1, wherein said fabric layer is a woven fabric.

3. The removable adhesive pant liner of claim 1, wherein said fabric layer is a knit fabric.

4. The removable adhesive pant liner of claim 3, wherein said knit fabric is a circular knit fabric providing fabric elasticity in both the longitudinal direction, the lateral direction and combination longitudinal and latitudinal directions.

5. The removable adhesive pant liner of claim 1, wherein said fabric layer is translucent.

6. The removable adhesive pant liner of claim 1, wherein said fabric layer comprises nylon fibers and Lycra fibers.

7. The removable adhesive pant liner of claim 4, wherein the nylon fibers comprise between 75 and 98 percent of said fabric layer.

8. The removable adhesive pant liner of claim 4, wherein the Lycra fibers comprise between 2 and 25 percent of said fabric layer.

9. The removable adhesive pant liner of claim 1, wherein the fabric layer consists of adsorbent fiber components.

10. The removable adhesive pant liner of claim 1, wherein a periphery of said fabric layer comprises edge finishing.

11. The removable adhesive pant liner of claim 1, wherein the first adhesive layer is on a portion of said bottom side of said fabric layer.

12. The removable adhesive pant liner of claim 1, wherein the first adhesive layer comprises a first adhesive that is a stronger adhesive than a second adhesive in said second adhesive layer.

13. The removable adhesive pant liner of claim 1, wherein said first adhesive layer covers a portion of said bottom side of said fabric layer.

14. The removable adhesive pant liner of claim 1, wherein said first adhesive layer does not cover a central portion of said bottom side of said fabric layer.

15. The removable adhesive pant liner of claim 1, wherein said paper layer comprises tissue paper.

16. The removable adhesive pant liner of claim 1, wherein said paper layer comprises a graphic design a top side of said tissue paper.

17. The removable adhesive pant liner of claim 1, wherein said paper layer comprises a graphic design on a bottom side of said tissue paper.

18. The removable adhesive pant liner of claim 1, wherein said paper layer comprises a graphic design.

19. The removable adhesive pant liner of claim 18, wherein said graphic design comprises said longitudinal and lateral direction stiffening region.

20. The removable adhesive pant liner of claim 1, wherein said paper layer comprises an alignment aide.

21. The removable adhesive pant liner of claim 1, wherein said paper layer comprises a z-fold in a longitudinal direction or a lateral direction.

22. The removable adhesive pant liner of claim 1, wherein said paper layer comprises a region of increased stiffness.

23. The removable adhesive pant liner of claim 22, wherein said region of increased stiffness comprises a stiffening agent material.

24. The removable adhesive pant liner of claim 22, wherein said region of increased stiffness region comprises channels formed in said paper layer.

25. The removable adhesive pant liner of claim 1, wherein said second adhesive layer substantially covers said bottom side of said paper layer.

26. The removable adhesive pant liner of claim 1, wherein said second adhesive layer is on a portion of said bottom side of said paper layer.

27. The removable adhesive pant liner of claim 1, wherein said second adhesive layer comprises a finger grip area without second adhesive, said finger grip being positioned at an edge of said removable adhesive pant liner.

28. The removable adhesive pant liner of claim 1, wherein said second adhesive layer does not cover at least a central portion of said bottom side of said paper layer.

29. The removable pant liner of claim 1, wherein said removable release layer is divided into a first and a second release layer portion.

30. The removable pant liner of clam 29, wherein said first and said second release layer portions are separated by a cut line that extends across said removable release layer.

31. The removable pant liner of claim 30, further comprising a first release layer tab on said first release layer portion, said first release layer tab being adjacent to said cut line and extending outside a periphery of said fabric layer.

32. The removable pant liner of claim 1, further comprising a removable cover layer on top of said fabric layer, said removable cover layer providing said longitudinal and lateral direction stiffening region.

33. The removable pant liner of claim 32, wherein said removable cover layer comprises a cover release layer and a third adhesive layer, said third adhesive layer being between said cover release layer and said fabric layer.

34. The removable pant liner of claim 33, wherein said adhesive layer is has a stronger adhesive attraction to said cover release layer than to said fabric layer.

35. The removable pant liner of claim 32, wherein said removable cover layer covers said fabric layer and is a contaminant barrier for said fabric layer.

36. The removable pant liner of claim 32, wherein said removable cover layer comprises a tab extending beyond a periphery of said fabric layer.

37. A removable pant liner comprising a plurality of layers, said removable pant liner comprising:
a top side having a predetermined shape, said top side having a longitudinal center line;
a crotch seam alignment aide that is visible from said top side of said removable pant liner, said crotch seam alignment aide being on said longitudinal center line, and
a garment adhesive layer that is removably attachable to a garment.

38. The removable pant liner of claim 37, wherein said crotch seam alignment aide is centered on said longitudinal center line.

39. The removable pant liner of claim 37, further comprising an adsorbent fabric layer.

40. The removable pant liner of claim 37, further comprising a knitted fabric layer that stretches in both a longitudinal direction along said longitudinal center line and a latitudinal direction that is substantially perpendicular to said longitudinal center line.

41. The removable pant liner of claim 37, further comprising a stiffening region extending at least in a lateral direction with respect to said longitudinal direction.

42. The removable pant liner of claim 37, further comprising wherein said garment adhesive layer comprises a finger grip region, said finger grip region is an area without a garment adhesive that is adjacent to the perimeter of said removable pant liner.

43. The removable pant liner of claim 37, wherein a portion of a central area of said of said garment adhesive layer does not have garment adhesive.

44. A removable liner for a crotch area of a pair of pants, said removable liner comprising:
a top side and a bottom side with a plurality of layers of materials in-between;
a fabric layer, being one of said plurality of layers, between said top side and said bottom side;
a garment adhesive layer, being one of said plurality of layers, below said fabric layer and between said top side and said bottom side, said garment adhesive layer being removably adherable to a garment;
a release layer, being one of said plurality of layers, below and adjacent to said garment adhesive layer, said release layer being removable;
a stiffening region, being part of at least one of said plurality of layers, said stiffening region maintains a stiffness in said removable liner when said release layer is removed.

45. The removable liner of claim 44, further comprising a crotch seam alignment aide that is visible on said top side of said removable liner.

46. The removable liner of claim 44, wherein said fabric layer is stretchable in more than one direction.

47. The removable liner of claim 44, wherein said stiffening region comprises a graphic design.

48. The removable liner of claim 44, wherein said garment liner further comprises a paper layer, being one of said plurality of layers, between said fabric layer and said garment adhesive layer.

49. The removable liner of claim 48, wherein said combination of said fabric layer, said paper layer, and said garment adhesive layer are limp.
